(19) United States
(12) Patent Application Publication
(10) Pub. No.: US 2022/0333077 A1
(43) Pub. Date: Oct. 20, 2022

(54) Product for Therapy and Methods

(71) Applicant: The University of Bristol, Bristo (GB)

(72) Inventors: Timothy James Satchwell, Bristol (GB); Ashley Mark Toye, Bristol (GB); Marjolein Meinders, Bristol (GB)

(73) Assignee: The University of Bristol, Bristol (GB)

(21) Appl. No.: 17/760,658

(22) PCT Filed: Sep. 21, 2020
(86) PCT No.: PCT/EP2020/076335

§ 371 Date: Mar. 15, 2022

(30) Foreign Application Priority Data
Sep. 20, 2019 (GB) 1913592.0

(51) Int. Cl. C12N 5/078; A61K 35/18; A61K 47/00

(52) U.S. Cl. CPC C12N 5/0641; A61K 35/18; A61K 47/00

(57) ABSTRACT

A method of making an erythroid cell comprising elevated levels of a target protein or polypeptide, the method comprising: a) provision of an erythroid progenitor which is able to express the target protein or polypeptide; b) expression of the target protein or polypeptide; and c) maturation of the erythroid progenitor into the erythroid cell; wherein during maturation of the erythroid progenitor into the erythroid cell, the target protein or polypeptide is configured and/or inhibited such that ubiquitination of the target protein or polypeptide is hindered or prevented. Erythroid cells, pharmaceutical compositions and methods of use related thereto, and a method of screening for proteins or polypeptides degraded by ubiquitination during maturation of an erythroid progenitor are also provided.

### TECHNICAL FIELD

This invention relates to erythroid cells comprising elevated levels of a target protein or polypeptide which can be used in therapy, and specifically to expressing protein or polypeptide in reticulocyte precursor cells and retaining high concentrations of the protein or polypeptide through differentiation. The invention relates to methods of manufacture, cell products, pharmaceutical compositions and therapeutic uses.

### BACKGROUND

A wide range of diseases are caused by protein or polypeptide defects or deficiency, such as enzyme defects or deficiency, and one approach to treatment of such diseases is use of protein or polypeptide replacement therapy. Enzyme replacement therapy, for example, involves administration of non-defective enzyme to the patient to compensate for the loss of function or loss of expression of the enzyme. Such administration can be used for more than just the restoration of function of defective or deficient proteins and polypeptides, and can for example provide entirely new functionalities to a subject. Examples include the use of exogenous proteins or polypeptides that treat conditions unrelated to defective proteins or polypeptides, such as use of exogenous enzymes that neutralise endogenous or exogenous toxins (for example, in removing a build-up of a certain metabolite or in counteracting a nerve agent or drug overdose).

A major challenge in protein or polypeptide administration is how to deliver the protein or polypeptide and ensure good bioavailability without eliciting a damaging immune response. One known approach for administration is to make use of a protein or polypeptide that is encapsulated by an erythrocyte, which can be prepared, for example, by inserting the protein or polypeptide into an erythrocyte by hypotonic lysis. The use of an erythrocyte vehicle allows for distribution of the protein or polypeptide around the body, while offering a level of protection of the enzyme from the subject's immune system and from the body's natural degradation pathways. The enzyme thymidine phosphorylase (TP) serves as an example for further explanation. Mitochondrial neurogastrointestinal encephalomyopathy (MNGIE) is an autosomal recessive metabolic disorder caused by mutations in the TYMP gene which encodes TP. Erythrocytes do not normally express TP, but hypotonic haemolysis and isotonic resealing can be used to encapsulate Escherichia coli TP in autologous red blood cells (Ihler, G. M. et al., Proc Natl Acad Sci USA, 1973, 70(9), p. 2663-2666; Bourgeaux, V., et al., Drug Des Devel Ther, 2016, 10, p. 665-676). This has been successfully used in the clinic, providing a proof-of-concept of achieving prolonged cessation of the MNGIE clinical phenotype by reducing plasma nucleoside levels (Bax, B. E., et al., Neurology, 2013, 81(14), p. 1269-71). Though this method works to some extent, the methodology of encapsulation within red blood cells using hypotonic lysis usually compromises cell membrane integrity and the life span of the red blood cells meaning frequent transfusions are required, and the patient can also develop antibodies against the bacterial enzyme (Levene, M., et al., Mol Ther Methods Clin Dev, 2018, 11, p. 1-8).

Another approach has been the use of genetic engineering to express the required enzyme within the cell. Mature red blood cells lack a nucleus and are therefore incapable of expressing an enzyme or enzymes, meaning any genetic engineering and protein expression must occur earlier in the lifetime of the cell, while it still has a nucleus. Advantages of this approach include retaining cell membrane integrity and providing the full lifespan of the red blood cell.

The present invention relates to an improved method for retaining a target protein or polypeptide throughout red blood cell development and maturation into the reticulocyte and/or erythrocyte to provide elevated concentrations of the target protein or polypeptide in the reticulocyte or erythrocyte. The method of the invention can therefore be used to retain a target protein or polypeptide in a reticulocyte and/or erythrocyte where previously the target protein or polypeptide would not be retained through the cell maturation process. Equally, where a target protein or polypeptide could be retained through maturation into the reticulocyte and/or erythrocyte, the method of the invention can be used to increase retention and increase the subsequent abundance of the target protein or polypeptide in the enucleated erythroid cell (reticulocyte and/or erythrocyte), allowing, for example, for reducing the dosage of the therapeutic.

### SUMMARY OF THE INVENTION

According to a first aspect, the invention provides a method of making a reticulocyte comprising elevated levels of a target protein or polypeptide, the method comprising: a) provision of an erythroid progenitor which is able to express the target protein or polypeptide; b) expression of the target protein or polypeptide; and c) maturation of the erythroid progenitor into the reticulocyte; wherein during maturation of the erythroid progenitor into the reticulocyte, the target protein or polypeptide is configured and/or inhibited such that ubiquitination of the target protein or polypeptide is hindered or prevented. As such, ubiquitin-mediated degradation of the target protein or polypeptide is hindered or prevented.

The inventors have observed that during the erythroid cell maturation and enucleation process, large quantities of certain proteins are removed through a highly active ubiquitination and ubiquitin-mediated degradation pathway. The inventors also identified that disruption of this ubiquitination process can be exploited to lead to much higher concentrations of a target protein or polypeptide being retained through the enucleation process and into the mature reticulocyte and subsequent erythrocyte. Specifically, the inventors have sought to hinder or prevent ubiquitination by interfering with the target protein or polypeptide (rather than interfering with ubiquitin ligases), by configuring and/or inhibiting the target protein or polypeptide. By localising the interference to the target protein or polypeptide, the inventors can also overcome issues associated with inhibiting ubiquitinase enzymes. Ubiquitination is an essential part of the erythroid cell maturation process (Nguyen, A. T., et al., Science, 2017, 357(6350)), therefore the global inhibition of ubiquitination using inhibitors such as MG132 is not a desirable solution to enhancing TP levels in reticulocytes. In the presence of a general ubiquitin ligase inhibitor many non-erythroid proteins would be retained in the developing reticulocyte or erythrocyte. As a result, many aspects of cell function would be disrupted which can adversely impact important aspects of the cell such as lifetime and immune system compatibility. In certain cases, broad ubiquitination inhibition may prevent differentiation altogether and/or lead to cell death. In contrast, with the present invention any configuration or inhibition occurs on the target protein or polypeptide and not on ubiquitin ligase enzymes, and therefore ubiquitin ligase enzyme function in respect of non-target proteins or polypeptides is not substantially impaired.

By “target protein or polypeptide”, we are generally referring to a target protein or a target polypeptide where elevated levels are retained in the enucleated erythroid cell (reticulocyte and/or erythrocyte) by means of the method of the invention. Three scenarios are envisioned for the target protein or polypeptide: (1) the target protein or polypeptide comprises a ubiquitination site; (2) the target protein or polypeptide is a variant of a source protein or polypeptide, wherein the source protein or polypeptide comprises a ubiquitination site, and wherein the target protein or polypeptide comprises one or more mutations with respect to the source protein that renders ubiquitination hindered or prevented; and/or (3) the target protein or polypeptide has no ubiquitination site. In scenario (1), ubiquitination can be hindered or prevented by provision of an inhibitor of the target protein or polypeptide ubiquitination site. In scenario (2), the protein or polypeptide expressed in the erythroid progenitor has a mutation that interferes with ubiquitination. The realisation of the importance of ubiquitination also provides for scenario (3), where an exogenous protein or polypeptide having no ubiquitination site is selected for expression.

By “elevated levels” of a target protein or polypeptide, we are generally referring to levels (or concentrations) of target protein or polypeptide in the reticulocyte (or subsequent erythrocyte) that are elevated with respect to the reticulocyte (or subsequent erythrocyte) where ubiquitination is not hindered or prevented. In the case of endogenous proteins or polypeptides (either naturally expressed or overexpressed) or exogenous proteins or polypeptides having a ubiquitination site, the levels are higher in the reticulocyte (or subsequent erythrocyte) when the ubiquitination site is inhibited during cell maturation than when the ubiquitination site is not inhibited during cell maturation. In the case of exogenous proteins or polypeptides comprising a mutation with respect to a source protein or polypeptide that renders ubiquitination hindered or prevented with respect to the source protein or polypeptide, the level is elevated with respect to the level of retention of the source protein or polypeptide. In the case of exogenous proteins having no ubiquitination site, the level in the reticulocyte (and subsequent erythrocyte) is greater than zero.

By “hindered”, we are generally referring to a lower level of ubiquitination and subsequent protein or polypeptide loss through the maturation of the erythroid progenitor as compared with maturation of the erythroid progenitor in the absence of the target protein or polypeptide being configured and/or inhibited such that ubiquitination is hindered. In other words, the likelihood of each protein or polypeptide molecule being ubiquitinated is reduced compared to an expected level of ubiquitination. This could be achieved through any method of interfering with ubiquitination for example by the provision of an inhibitor and/or through provision of an erythroid progenitor that has undergone a genetic modification to hinder ubiquitination. By “prevented”, we are generally referring to ubiquitination not being possible. For example, this could be through deletion of the ubiquitination site or through provision of a protein or polypeptide that has no ubiquitination site. For instance, ubiquitination of the target protein or polypeptide can be hindered or prevented by: i) where the target protein or polypeptide comprises a ubiquitination site, provision of an inhibitor of the target protein or polypeptide ubiquitination site during maturation of the erythroid progenitor into the reticulocyte; ii) where the target protein or polypeptide is a variant of a source protein or polypeptide with a ubiquitination site, provision of an erythroid progenitor which is able to express a target protein or polypeptide comprising a mutation that renders ubiquitination hindered or prevented with respect to the source protein or polypeptide; and/or iii) provision of an erythroid progenitor which is able to express an exogenous protein or polypeptide having no ubiquitination site.

The erythroid progenitor is preferably able to express the target protein or polypeptide. By this, we mean that the erythroid progenitor contains genetic material that expresses the target protein or polypeptide. As such, the target protein or polypeptide does not need to be inserted into the cell post-enucleation and techniques such as hypotonic lysis can be avoided.

The erythroid cell (e.g. erythroid progenitor, reticulocyte and/or erythrocyte) may comprise more than one type of target protein or target polypeptide. In such an embodiment, multiple protein components can be used, for example, to provide a chain of enzymes in order to catalyse a sequence of reactions.

Where a protein or peptide has more than one ubiquitination site, at least one is hindered or prevented from being ubiquitinated. In a preferred embodiment, all ubiquitination sites are hindered or prevented from being ubiquitinated.

Where an inhibitor is used, it is provided during maturation of the erythroid progenitor into the reticulocyte and/or erythrocyte. By this, we mean that the inhibitor is provided during at least part of the differentiation and maturation process, typically at least the later part of the maturation process. For example, the inhibitor may only be added during the terminal differentiation stage. It is preferred that the inhibitor is present during enucleation, and preferably for the whole duration of enucleation. As such, the inhibitor may be added immediately prior to enucleation, such as in the culture medium used to start enucleation, or during the stage in the differentiation lineage immediately prior to enucleation. In one example, the inhibitor is added at the polychromatic erythroblast stage. In other words, the inhibitor does not necessarily have to be present throughout the whole erythroid maturation process. For example, the inhibitor may be added only in the later stages of the maturation process which is particularly beneficial if the inhibitor has deleterious effects on cell proliferation or survival if added earlier.

The target protein or polypeptide may be a variant of a source protein or polypeptide with a ubiquitination site, wherein the variant has a mutation or mutations with respect to the source protein or polypeptide that renders ubiquitination hindered or prevented with respect to the source protein or polypeptide. The mutation can be to a single amino acid or multiple amino acids. The mutation can encompass point mutations, insertions or deletions. The mutation can be to consecutive or non-consecutive amino acids or stretches of amino acids. In one embodiment, the mutation may sterically block the ubiquitin ligase from delivering ubiquitin to the ubiquitination site. Alternatively, the mutation may disrupt the configuration of the ubiquitination site such that the ubiquitinase is no longer able to bind ubiquitin to the ubiquitination site, for example by preventing the ubiquitin ligase from recognising the ubiquitination site. This mutation could be within the ubiquitination site, particularly the amino acid which is ubiquitinated, or could be at another position within the protein or polypeptide that nonetheless disrupts the ubiquitination site. This could also involve deletion of portions of the ubiquitination site, particularly the amino acid which is ubiquitinated, or deletion of the entire ubiquitination site consensus sequence. In any event, modification of the amino acid sequence must be such that it does not destroy the required activity of the protein or polypeptide.

In certain proteins or polypeptides, one or more post-translational modifications are required before the protein or polypeptide can be ubiquitinated. For example, acetylation, methylation, phosphorylation, glycosylation and/or lipidation may be required before ubiquitination can occur. In these cases, the mutation that renders ubiquitination hindered or prevented can be a mutation that hinders or prevents post-translational modification. For example, the enzyme glutamine synthase requires acetylation prior to being ubiquitinated. Disruption of the acetylation and/or ubiquitination sites can therefore be used to disrupt ubiquitination. One technique for removing the acetylation site of glutamine synthase is to remove the N-terminus, for example by removing at least 5, 10, 15 or 20 amino acids from the N-terminus and up to 30, 40, 50 or 60 amino acids from the N-terminus. It is possible that reductions in activity of the protein or polypeptide may be tolerable if the quantity of protein or polypeptide that survives erythroid cell maturation is sufficient to compensate.

Typically, it is envisioned that erythroid cells will be brought to the reticulocyte stage and put into storage at the reticulocyte stage. Reticulocytes are not fully matured erythrocytes, but are enucleated. It is envisioned that the reticulocyte cells will also be used for administration to a subject and allowed to mature to erythrocyte cells in the body of the subject as this ensures the full lifetime of the erythrocyte cell is available. Once injected into the body, reticulocytes take around 1-2 days to mature to erythrocytes and from this point the erythrocyte still has its full lifetime. However, it is also possible that the cells will be fully matured to the erythrocyte cell prior to administration to a subject.

As such, in an embodiment the method of the first aspect of the invention includes the further step of maturation of the reticulocyte into an erythrocyte. The maturation of the reticulocyte into an erythrocyte involves a further stage of removing proteins (and further material) as the cell turns from the reticulocyte to the biconcave erythrocyte. Improved protein or polypeptide retention is expected from continuing (or initiating) the hindrance or prevention of ubiquitination of the target protein or polypeptide through this further step of maturation.

A second aspect of the invention provides a method of making an erythrocyte comprising a target protein or polypeptide, the method comprising steps (a)-(c) of the first aspect of the invention, and comprising the further step of: d) red cell development, in particular maturation of the reticulocyte into the erythrocyte; wherein during maturation to the erythrocyte, the target protein or polypeptide is configured and/or inhibited such that ubiquitination is hindered or prevented, preferably hindered or prevented by any of (i)-(iii) of the first aspect of the invention.

In a preferred embodiment, the method of making a reticulocyte and/or erythrocyte is an in vitro method.

The target protein can comprise an endogenous protein and/or the target polypeptide can comprise an endogenous polypeptide. Here, we are generally referring to endogenous protein or polypeptide that is not artificially overexpressed. In other words, this refers to endogenous proteins or polypeptides at endogenous concentrations prior to maturation of the erythroid progenitor. The invention allows for elevating the levels of such endogenous proteins and polypeptides in the reticulocyte or erythrocyte by hindering or preventing ubiquitination of the proteins or polypeptides, typically wherein the protein or polypeptide has a ubiquitination site and the hindering or preventing ubiquitination is achieved with an inhibitor.

The target protein can comprise an overexpressed endogenous protein or exogenous protein and/or the target polypeptide can comprise an overexpressed endogenous polypeptide or exogenous polypeptide.

By “overexpressed” we are generally referring to endogenous proteins or polypeptides which are artificially elevated to higher concentrations in the erythroid progenitor, prior to cell maturation and/or enucleation. Overexpression techniques are well-known in the art and include insertion of further copies of the gene and/or provision or expression of appropriate transcription regulators. For instance, one suitable technique is to use a transcription factor or other enhancer to increase the expression of the protein (where the protein can be encoded by the cell's own DNA or by added DNA), such as by using a CRISPR enhancer and a specific guide to increase levels of expression. Suitable gene manipulation techniques are well-known in the art and include the use of techniques such as CRISPR-Cas9 base editing as well as lentiviral vector gene insertion.

Gene insertion would generally be conducted at one of the differentiation stages prior to enucleation. For instance, FIG. 7 shows a schematic of cell differentiation lineages, starting from a hematopoietic stem cell and from a BEL-A pro-erythroblast respectively, exemplifying stages in vitro where a viral vector (such as is used for lentiviral vector gene insertion) can be added. In the case of the CD34+ cells, in particular, it is possible to add virus at later steps, i.e. up to just before differentiation. A benefit of adding virus earlier is that you need less virus and reagents.

By “exogenous” we are generally referring to proteins or polypeptides which are not naturally expressed in the erythroid progenitor. This includes proteins or polypeptides which comprise one or more amino acid mutations with respect to the endogenous protein. This covers, for example, therapy where the erythroid progenitor expresses a defective protein or polypeptide, and the exogenous enzyme is the functional protein or polypeptide. Exogenous also covers isoforms from other cells or species, or non-naturally occurring proteins or polypeptides, such as hybrid proteins, chimeric proteins, fusion proteins or de novo protein or polypeptide sequences. As set out above, gene manipulation techniques that can be used for expression of exogenous proteins or polypeptides are well-known in the art.

The erythroid progenitor can be autologous. For example, the erythroid progenitor can be removed from the subject, genetically manipulated to express the target protein or polypeptide, and ultimately returned to the subject. Preferably, the erythroid progenitor is allogeneic but would be applied to a suitable subject who had matched blood groups.

The inhibitor can be a natural substrate or a natural product of the target protein or polypeptide. This applies where the ubiquitination site lies within the active site of the protein or polypeptide. This is a mechanism for preventing degradation of the protein or polypeptide by occupying the active site and thereby preventing degradation. This is sometimes termed “substrate masking” because the substrate obscures accessibility to the ubiquitination site, and can be exploited to prevent degradation through the cell maturation process by supplying elevated levels of a suitable natural substrate or product. Typically, the protein or polypeptide in this case will be an enzyme.

Equally, the inhibitor can be a reversible inhibitor of a natural substrate or natural product of the target protein or polypeptide. Such reversible inhibitors include derivatives and analogues (i.e. chemical mimics) of the natural substrate or product, that typically would not be turned over by the protein or polypeptide.

The erythroid progenitor is not particularly limited and can be any cell that is capable of maturing into a reticulocyte and subsequently an erythrocyte. The term “erythroid progenitor” can be used to refer to cells at different stages along the maturation/differentiation pathway. The term “erythroid progenitor” typically refers to cells that have a cell nucleus, i.e. before enucleation has started. The erythroid progenitor can be a stem cell, haematopoietic stem cell, induced pluripotent cell, erythroid immortalized cell line or erythroblast cell. Preferably the erythroid progenitor is a CD34+ cell, CD34− cell, or a BEL-A cell.

By “enucleated erythroid cell”, we are generally referring to a cell derived from an erythroid progenitor that has undergone enucleation. Typical examples of such a cell include a reticulocyte or erythrocyte. Enucleated erythroid cells display erythroid band 3 (Anion exchanger 1 (AE1); solute carrier family 4 member 1; SLC4A1) protein on the erythroid cell surface. In production of enucleated erythroid cells according to the invention, the enucleated erythroid cell may have further proteins engineered out (e.g. haemoglobin or blood group proteins), but the enucleated erythroid cell can still be identified as such by the presence of erythroid band 3/AE1 and by having no nucleus. For instance, the reticulocytes or erythrocytes of the invention may have proteins such as haemoglobin or blood group proteins engineered out but can still be identified as such by the presence of erythroid band 3/AE1 and by having no nucleus. Such cells may be further identified by the presence of erythroid spectrin based cytoskeleton.

Preferably, the target protein or polypeptide is a therapeutic protein or polypeptide. Preferably, the target protein or polypeptide is an enzyme. In a particular embodiment, the target protein is thymidine phosphorylase, glutamine synthase, hexokinase, glucokinase, phenylalanine hydroxylase, alcohol dehydrogenase, catalase, glucose-6-phosphate dehydrogenase, adenosine deaminase, asparaginase, uricase, bacterial L-phenylalanine ammonia lyase, alanine aminotransferase, glutamate dehydrogenase, arginine deiminase, or arginase. Preferably the target protein is thymidine phosphorylase, glutamine synthase, hexokinase, glucokinase, phenylalanine hydroxylase, alcohol dehydrogenase, catalase, glucose-6-phosphate dehydrogenase, adenosine deaminase, asparaginase, uricase or bacterial L-phenylalanine ammonia lyase. More preferably the target protein is thymidine phosphorylase.

Using thymidine phosphorylase (TP) as an example protein, initial experiments revealed that inhibition of ubiquitination with MG132 protected TP from degradation during differentiation (see Example 3). This shows that inhibition of ubiquitination can successfully be used to prevent TP degradation. In this particular example, mutation of the ubiquitination site adversely affected the desired enzyme activity. Therefore, in editing out a ubiquitination site, consideration should be given to effects on the desired protein activity. For example, the enzyme active site could be reengineered to retain activity while removing the ubiquitination sites or an alternate means of disrupting TP ubiquitination could be used. For instance, the inventors identified that the TP ubiquitination site is part of the active site, and therefore the substrate thymidine could actually block the ubiquitination site. Based on this, the inventors then demonstrated that blocking the (un-mutated) ubiquitination site with thymidine added to the media led to roughly double the concentration of active TP in the reticulocyte (see Example 5). Further detail can be found in Meinders et al., Molecular Therapy—Methods & Clinical Development, volume 17, page 822-830, Jun. 12, 2020 (published after the priority date of the present application), the content of which is incorporated herein by reference in its entirety. The thymidine phosphorylase ubiquitination site can be inhibited by thymidine, deoxyuridine, thymine, uridine, 2-deoxy ribose 1-phosphate, or derivatives or analogs of these. Preferably, the thymidine phosphorylase ubiquitination site is inhibited with thymidine.

This proof of concept also provides evidence that disruption of the ubiquitination site by other means, such as by appropriate engineering of the protein sequence as discussed above, can lead to the same result. For instance, sequence analysis suggests that glutamine synthase has ubiquitination sites that are not in the active site. This means that the substrate masking technique is unlikely to be suitable for hindering ubiquitination of glutamine synthase. However, this separate location of the ubiquitination sites to the active site means that mutations can be made that are likely to disrupt ubiquitination without affecting activity, meaning such modifications render the enzyme capable of being retained throughout differentiation and enucleation of an erythrocyte progenitor and further increase levels in the enucleated erythroid cells.

In this way, the substrate masking technique and mutation technique provide alternative solutions that can be selected depending on where the ubiquitination site is located within the target protein or polypeptide.

Where the target protein is an exogenous protein or polypeptide having no ubiquitination site, in one embodiment it is a non-eukaryotic protein, preferably a bacterial protein or polypeptide. Such proteins and polypeptides from non-eukaryotic sources such as bacteria, in their wild-type form, typically do not have ubiquitination sites. When the target protein or polypeptide is bacterial, this includes the wild-type bacterial amino acid sequence. As is known in the art, when expressing a bacterial protein in a human cell, certain codons that encode for specific amino acids in a bacterial expression system should be converted to the equivalent human codons for expression of those specific amino acids. This is readily conducted using known molecular biology principles. Changes to the bacterial amino acid sequence may also be useful for bringing about certain effects, such as optimizing expression in a human expression system. Where the bacterial amino acid sequence is modified, the sequence should still produce the intended function, particularly without the reintroduction of ubiquitination sites.

The inventors have identified that bacterial uricase (codon optimized for expression in a human expression system) is indeed retained in high levels through the enucleation process into the enucleated erythroid cell. The inventors have therefore identified that the pool of proteins and polypeptides that naturally lack a ubiquitination site can be targeted to identify proteins and polypeptides that are particularly suited to the method of the present invention. In particular, the present inventors have identified that bacterial proteins provide a viable pool of proteins and polypeptides that can be employed with the method of the current invention to provide enucleated erythroid cells with high levels of protein or polypeptide for use in therapy.

In one embodiment the target protein is thymidine phosphorylase. Where thymidine phosphorylase is selected as the target protein, the thymidine phosphorylase ubiquitination site is preferably inhibited by thymidine, deoxyuridine, thymine, uridine, 2-deoxy ribose 1-phosphate, or derivatives or analogs of these. Preferably, the thymidine phosphorylase ubiquitination site is inhibited with thymidine. Alternative approaches could involve careful protein engineering to disrupt the ubiquitination site without preventing enzyme activity, or expression of a bacterial version of thymidine phosphorylase which exploits the fact that bacterial enzymes lack ubiquitination sites while catalyzing the same enzymatic reaction. Where the target protein is thymidine phosphorylase, the enucleated erythroid cell (reticulocyte and/or erythrocyte) is preferably for use in the treatment of Mitochondrial Neurogastrointestinal Encephalomyopathy.

In one embodiment the target protein is glutamine synthase. We have shown that human glutamine synthase can be overexpressed using lentivirus and application of ubiquitination inhibitor MG132 increases glutamine synthase levels in reticulocytes (example 6, FIG. 8). The strategies of the invention, which are targeted to the glutamine synthase rather than the ubiquitinase, are therefore expected to produce improved expression and retention of glutamine synthase in a healthy enucleated cell. As set out above, sequence analysis suggests that glutamine synthase ubiquitination sites are not in the active site. Expression of glutamine synthase comprising a mutation that renders ubiquitination hindered or prevented with respect to the source protein or polypeptide should therefore be suitable for improving glutamine synthase retention through maturation to the enucleated erythroid cell. Glutamine synthase may require acetylation prior to being ubiquitinated (Van Nguyen et al., Molecular Cell (2016) 61 (6):809-820). As such, disruption of the acetylation and/or ubiquitination sites can therefore be used as a further technique to disrupt ubiquitination. One technique for removing the acetylation site of glutamine synthase is to remove the N-terminus, for example by removing at least 5, 10, 15 or 20 amino acids from the N-terminus and up to 30, 40, 50 or 60 amino acids from the N-terminus. Alternatively, a bacterial version of glutamine synthase may be overexpressed which naturally does not contain ubiquitination sites or may be engineered not to contain such sites. Where the target protein is glutamine synthase, the enucleated erythroid cell (reticulocyte and/or erythrocyte) is preferably for use in the treatment of hyperammonemia.

In one embodiment, the target protein is a hexokinase such as glucokinase. Glucokinase is a liver and pancreas enzyme, which has a high Kₘ for glucose. Glucokinase is a hexokinase isoenzyme and homologous to three other hexokinases. It is known that glucokinase is regulated by ubiquitination (see e.g. Hofmeister-Brix et al., Biochem 3 (2013) 456 (2): 173-184). Glucokinase is therefore particularly suited to improvement of retention through enucleation by mutation to render ubiquitination hindered or prevented. Alternatively, a bacterial version of hexokinase or glucokinase may be overexpressed which naturally does not contain ubiquitination sites or may be engineered not to contain such sites. Where the target protein is hexokinase and/or glucokinase, the enucleated erythroid cell (reticulocyte and/or erythrocyte) is preferably for use in the treatment of hyperglycaemia.

In one embodiment the target protein is phenylalanine hydroxylase. It is known that recombinant phenylalanine hydroxylase is ubiquitinated (see e.g. Doskeland and Flatmark, Biochem J. 1996 Nov. 1; 319(Pt 3): 941-945). In particular, recombinant protein was ubiquitinated by cell lysates on specific lysines. Misfolded protein has increased ubiquitination and degradation. This evidences that phenylalanine hydroxylase is suited to improvement of retention through enucleation by mutation to render ubiquitination hindered or prevented. Alternatively, a bacterial version of phenylalanine hydroxylase may be overexpressed which naturally does not contain ubiquitination sites or may be engineered not to contain such sites. Where the target protein is phenylalanine hydroxylase, the enucleated erythroid cell (reticulocyte and/or erythrocyte) is preferably for use in the treatment of phenylalanine hydroxylase enzyme deficiency.

In one embodiment the target protein is alcohol dehydrogenase. It is known that alcohol dehydrogenase is ubiquitinated (see e.g. Mezey et al., Biochem Biophys Res Commun, Volume 285, Issue 3, 20 Jul. 2001, Pages 644-648). Alcohol dehydrogenase is therefore suited to improvement of retention through enucleation by mutation to render ubiquitination hindered or prevented. Alternatively, another alcohol dehydrogenase isoform or a bacterial version of alcohol dehydrogenase could be overexpressed which naturally do not contain ubiquitination sites or may be engineered not to contain such sites. Where the target protein is alcohol dehydrogenase, the enucleated erythroid cell (reticulocyte and/or erythrocyte) is preferably for use in the treatment of alcohol toxicity/detoxification.

In one embodiment, the target protein is catalase. It is known that catalase degradation is regulated by ubiquitination which is instigated by tyrosine phosphorylation (see e.g. Cao et al, Biochemistry, 2003 Sep. 9, 42(35), 10348-53). Catalase is therefore suited to improvement of retention through enucleation by mutation to render ubiquitination hindered or prevented. Alternatively, a bacterial version of catalase may be overexpressed which naturally does not contain ubiquitination sites or may be engineered not to contain such sites. Where the target protein is catalase, the enucleated erythroid cell (reticulocyte and/or erythrocyte) is preferably for use in the treatment of catalase deficiency and/or preventing cell damage by reactive oxygen species.

In one embodiment, the target protein is glucose-6-phosphate dehydrogenase (G6PD). There is evidence of ubiquitination and degradation of G6PD in human podocytes (see e.g. Wang et al., The FASEB Journal, 2019, 33:5, 6296-6310). G6PD is therefore suited to improvement of retention through enucleation by mutation to render ubiquitination hindered or prevented. Alternatively, a bacterial version of G6PD may be overexpressed which naturally does not contain ubiquitination sites or may be engineered not to contain such sites. Where the target protein is G6PD, the enucleated erythroid cell (reticulocyte and/or erythrocyte) is preferably for use in the treatment of G6PD deficiency.

In one embodiment the target protein is adenosine deaminase. As set out above, sequence analysis suggests that adenosine deaminase ubiquitination sites are not in the active site. We have shown that adenosine deaminase is retained through enucleation into reticulocytes (FIG. 9). Expression of adenosine deaminase comprising a mutation that renders ubiquitination hindered or prevented with respect to the source protein or polypeptide is therefore expected to improve adenosine deaminase retention through enucleation and lead to increased concentrations of adenosine deaminase in the enucleated erythroid cell. Alternatively, a bacterial version of adenosine deaminase may be overexpressed which naturally does not contain ubiquitination sites or may be engineered not to contain such sites. Where the target protein is adenosine deaminase, the enucleated erythroid cell (reticulocyte and/or erythrocyte) is preferably for use in the treatment of adenosine deaminase deficiency

In one embodiment, the target protein is asparaginase or L-asparaginase. Sequence analysis suggests that L-asparaginase ubiquitination sites exist and are not in the active site. We have shown that L-asparaginase is retained through enucleation into reticulocytes (FIG. 10). Expression of L-asparaginase comprising a mutation that renders ubiquitination hindered or prevented with respect to the source protein or polypeptide is therefore expected to improve L-asparaginase retention through enucleation and lead to increased concentrations of L-asparaginase in the enucleated erythroid cell. Alternatively, a bacterial version of L-asparaginase may be overexpressed which naturally does not contain ubiquitination sites or may be engineered not to contain such sites. Where the target protein is asparaginase or L-asparaginase, the enucleated erythroid cell (reticulocyte and/or erythrocyte) is preferably for use in the treatment of acute lymphoblastic leukemia cancer.

In one embodiment, the target protein is uricase, preferably bacterial uricase. The human version of uricase is known to be non-functional. Therefore, preferably, the uricase is bacterial uricase which has no detectable ubiquitination sites or could be a version of the human uricase with mutations added to reactivate the enzyme activity and remove ubiquitination, or version taken from another species which have had the ubiquitination sites removed. We have established that bacterial uricase (codon-optimised for human expression) expresses well in erythroid progenitor cells and is retained well through enucleation into the reticulocyte (FIG. 11). Where the target protein is uricase, the enucleated erythroid cell (reticulocyte and/or erythrocyte) is preferably for use in the treatment of hyperuricemia.

In one embodiment, the target protein is bacterial L-phenylalanine ammonia lyase (PAL). Bacterial PAL is known for use in treatment (see Sarkissian et al, PNAS Mar. 2, 1999 96 (5) 2339-2344) and naturally has no ubiquitination site. Where the target protein is bacterial PAL, the enucleated erythroid cell (reticulocyte and/or erythrocyte) is preferably for use in the treatment of phenylketonuria/phenylalanine hydroxylase enzyme deficiency.

In one embodiment, both alanine aminotransferase and glutamate dehydrogenase are expressed in the erythroid precursor and are retained through to the enucleated erythroid cell. In this embodiment, the target protein can be alanine aminotransferase or glutamate dehydrogenase. In this embodiment, one protein is subject to the method of the invention and the other is carried through enucleation without affecting ubiquitination. In a preferred embodiment, the target protein is alanine aminotransferase and a further target protein is glutamate dehydrogenase. By this, we mean that both proteins are expressed in the erythroid precursor and both proteins are subject to methods of the invention to improve retention through to the enucleated erythroid cell. Regarding alanine aminotransferase, the inventors conducted a site scan for ubiquitin consensus sequences and identified ubiquitin sites on both human isoforms. Expression alanine aminotransferase comprising a mutation that renders ubiquitination hindered or prevented with respect to the source protein or polypeptide is therefore expected to improve alanine aminotransferase retention through enucleation and lead to increased concentrations of alanine aminotransferase in the enucleated erythroid cell. Alternatively, a bacterial version of alanine aminotransferase may be overexpressed which naturally does not contain ubiquitination sites or may be engineered not to contain such sites. Regarding glutamate dehydrogenase, the inventors conducted a site scan for ubiquitin consensus sequences and identified ubiquitin sites on one of two isoforms. Expression of ubiquitin-taggable glutamate dehydrogenase comprising a mutation that renders ubiquitination hindered or prevented with respect to the source protein or polypeptide is therefore expected to improve glutamate dehydrogenase retention through enucleation and lead to increased concentrations of glutamate dehydrogenase in the enucleated erythroid cell. Alternatively, the isoform lacking ubiquitin sites or a bacterial version of glutamate dehydrogenase may be overexpressed which naturally does not contain ubiquitination sites or may be engineered not to contain such sites. Where the target protein is alanine aminotransferase and/or the further target protein is glutamate dehydrogenase, the enucleated erythroid cell (reticulocyte and/or erythrocyte) is preferably for use in the treatment of hyperammonaemia.

In one embodiment, the target protein is arginine deiminase. The inventors conducted a site scan for ubiquitin consensus sequences and identified ubiquitin sites on three of six isoforms. Expression of ubiquitin-taggable arginine deiminase comprising a mutation that renders ubiquitination hindered or prevented with respect to the source protein or polypeptide is therefore expected to improve arginine deiminase retention through enucleation and lead to increased concentrations of arginine deiminase in the enucleated erythroid cell. Alternatively, an isoform lacking ubiquitin sites or a bacterial version of arginine deiminase may be overexpressed which naturally does not contain ubiquitination sites or may be engineered not to contain such sites. Where the target protein is arginine deiminase, the enucleated erythroid cell (reticulocyte and/or erythrocyte) is preferably for use in the treatment of cancer.

In one embodiment, the target protein is arginase. The inventors conducted a site scan for ubiquitin consensus sequences and identified ubiquitin sites on one isoform. Expression of the ubiquitin-taggable arginase isoform comprising a mutation that renders ubiquitination hindered or prevented with respect to the source protein or polypeptide is therefore expected to improve arginase retention through enucleation and lead to increased concentrations of arginase in the enucleated erythroid cell. Alternatively, the isoform lacking ubiquitin sites or a bacterial version of arginase may be overexpressed which naturally does not contain ubiquitination sites or may be engineered not to contain such sites. Where the target protein is arginase, the enucleated erythroid cell (reticulocyte and/or erythrocyte) is preferably for use in the treatment of hyperargininaemia.

In one embodiment, the reticulocyte or erythrocyte is an isolated reticulocyte or isolated erythrocyte. By “isolated” we are generally referring to the reticulocyte or erythrocyte being isolated from the body of a subject or patient. In other words, the reticulocyte or erythrocyte is produced in vitro. As such, the isolated reticulocyte or isolated erythrocyte can be an isolated reticulocyte cell population or isolated erythrocyte cell population and can be in the presence of other cells or biological material, particularly biological material and chemical factors required to promote cell maturation and/or to facilitate cell storage.

According to a third aspect, the invention provides an erythroid cell comprising a target protein or polypeptide, wherein: i) where the target protein or polypeptide comprises a ubiquitination site, the erythroid cell further comprises an inhibitor of the target protein or polypeptide ubiquitination site; and/or ii) where the target protein or polypeptide is a variant of a source protein or polypeptide with a ubiquitination site, the target protein or polypeptide comprises a mutation that renders ubiquitination hindered or prevented with respect to the source protein or polypeptide; and/or iii) the target protein or polypeptide comprises an exogenous protein or polypeptide having no ubiquitination site.

By “erythroid cell”, we are generally referring to an erythroid progenitor, reticulocyte precursor, reticulocyte or erythrocyte, preferably a reticulocyte or erythrocyte, more preferably a reticulocyte. In one embodiment, the erythroid cell is an erythroid progenitor cell. In another embodiment, the erythroid cell is an enucleated erythroid cell (such as a reticulocyte or erythrocyte).

When an enucleated erythroid cell is produced by hindering or preventing ubiquitination of the target protein or polypeptide by a method according to the invention, the erythroid cell will contain elevated levels of the target protein or polypeptide than compared with an enucleated erythroid cell that is produced by a method that is identical in all respects except that ubiquitination of the target protein or polypeptide is unhindered.

Where the inhibitor is an endogenous substance, this refers to elevated levels of inhibitor.

In one embodiment, the target protein can comprise an endogenous protein and/or the target polypeptide can comprise an endogenous polypeptide. In another embodiment, the target protein can comprise an overexpressed endogenous protein or exogenous protein and/or the target polypeptide can comprise an overexpressed endogenous polypeptide or exogenous polypeptide.

The inhibitor can be a natural substrate or a natural product of the target protein or polypeptide, or can be a reversible inhibitor of a natural substrate or natural product of the target protein or polypeptide.

In one embodiment, the target protein is an enzyme. In one particular embodiment, the target protein is thymidine phosphorylase, glutamine synthase, hexokinase/glucokinase, phenylalanine hydroxylase, alcohol dehydrogenase, catalase, glucose-6-phosphate dehydrogenase, adenosine deaminase, L-asparaginase, uricase, bacterial L-phenylalanine ammonia lyase, alanine aminotransferase, glutamate dehydrogenase, arginine deiminase, or arginase. Preferably the target protein is thymidine phosphorylase, glutamine synthase, hexokinase/glucokinase, phenylalanine hydroxylase, alcohol dehydrogenase, catalase, glucose-6-phosphate dehydrogenase, adenosine deaminase, L-asparaginase, uricase or bacterial L-phenylalanine ammonia lyase. More preferably the target protein is thymidine phosphorylase.

When the target protein is thymidine phosphorylase, the thymidine phosphorylase ubiquitination site can be inhibited by thymidine, deoxyuridine, thymine, uridine and/or 2-deoxy ribose 1-phosphate, or derivatives or analogues of these, preferably thymidine.

The erythroid cell can be an isolated erythroid cell.

In one embodiment, the target protein or polypeptide that has no ubiquitination site can be a non-eukaryotic protein, preferably a bacterial protein.

According to a fourth aspect, the invention provides an erythroid cell obtainable by the method of the first or second aspects of the invention.

According to a fifth aspect, the invention provides a pharmaceutical composition comprising an erythroid cell according to third or fourth aspects of the invention and a pharmaceutically acceptable carrier, excipient and/or adjuvant. Preferably, the pharmaceutical composition comprises an erythroid cell stored in an erythroid cell storage buffer further comprising the inhibitor of the target protein or polypeptide. This applies in the situation where the target protein or polypeptide comprises a ubiquitination site and the erythroid cell further comprises an inhibitor of the target protein or polypeptide ubiquitination site. Storage can be short-term storage, long-term storage, cryogenic storage, storage in the clinic, storage during transportation, or any other storage up until the point of administration to a subject.

According to a sixth aspect, the invention provides a pharmaceutical composition comprising an erythroid cell comprising a target protein or polypeptide comprising a ubiquitination site and an inhibitor of the target protein or polypeptide ubiquitination site and a pharmaceutically acceptable carrier, excipient and/or adjuvant. Typically, the inhibitor will be an exogenous inhibitor, i.e. an inhibitor that is added to the erythroid cell. Where the inhibitor is naturally present in the erythroid cell, we are generally referring to artificially elevated levels of inhibitor. In a preferred embodiment, the pharmaceutical composition according to the sixth aspect of the invention comprises an erythroid cell stored in an erythroid cell storage buffer comprising the inhibitor of the target protein or polypeptide ubiquitination site. The erythroid cell in an erythroid cell storage buffer will typically be for short-term storage, long-term storage, cryogenic storage, storage in the clinic, storage during transportation, or any other storage up until the point of administration to a subject.

According to a seventh aspect, the invention provides a pharmaceutical composition comprising an erythroid cell comprising a target protein or polypeptide, wherein the target protein or polypeptide is a variant of a source protein comprising a ubiquitination site, and wherein the target protein or polypeptide comprises a mutation that renders ubiquitination hindered or prevented with respect to the source protein or polypeptide, and a pharmaceutically acceptable carrier, excipient and/or adjuvant.

The compositions of the invention may comprise excipients or pharmaceutically acceptable adjuvants, carriers or fillers as would be understood in the art, as well as reagents such as stabilisers, antimicrobial agents, cryoprotectants, antioxidants, free radical scavengers, solubilizing agents, tonicifying agents, and surfactants. In a preferred embodiment, the pharmaceutical composition is stored with the cells at the reticulocyte stage in a suitable reticulocyte storage medium. Suitable mediums are known in the art and include SAGM, PAGGM, AS1, AS3, human plasma or artificial plasma solution, as well as physiologically administrable buffers such as phosphate buffered saline, or mixtures of any of these.

According to an eighth aspect, the invention provides an erythroid cell according to the third or fourth aspects of the invention, or a pharmaceutical composition according to the fifth, sixth or seventh aspects of the invention, for use in therapy. Preferably any erythroid cells used in therapy are enucleated cells. One advantage of using enucleated cells in therapy is that any genetically modified material has been expelled during enucleation and as such there are no concerns around administration of genetically modified material to a subject.

In one embodiment, the use is use in enzyme replacement therapy, organ reconditioning or detoxification, preferably the treatment of Mitochondrial Neurogastrointestinal Encephalomyopathy, hyperammonemia, Hyperglycaemia, phenylalanine hydroxylase enzyme deficiency, alcohol toxicity/detoxification, catalase deficiency and/or preventing cell damage by reactive oxygen species, G6PD deficiency, adenosine deaminase deficiency, acute lymphoblastic leukemia cancer, hyperuricemia, phenylketonuria/phenylalanine hydroxylase enzyme deficiency, or hyperargininaemia. Preferably the use is in treatment of Mitochondrial Neurogastrointestinal Encephalomyopathy, Hyperammonemia, Hyperglycaemia, phenylalanine hydroxylase enzyme deficiency, alcohol toxicity/detoxification, catalase deficiency and/or preventing cell damage by reactive oxygen species, G6PD deficiency, or hyperuricemia.

According to a ninth aspect, the invention provides the use of an erythroid cell according to third or fourth aspects of the invention, or a pharmaceutical composition according to the fifth, sixth or seventh aspects of the invention, for the manufacture of a medicament for use in therapy.

According to a tenth aspect, the invention provides a method of treatment of a subject in need thereof, the method comprising administering a pharmaceutically effective amount of an erythroid cell according to the third or fourth aspects of the invention, or a pharmaceutically effective amount of a pharmaceutical composition according to the fifth, sixth or seventh aspects of the invention.

According to an eleventh aspect, the invention provides a method of screening for a protein or polypeptide that is degraded by ubiquitination during maturation of an erythroid progenitor, the method comprising expressing a test protein in an erythroid progenitor and: a) identifying if the test protein amount is elevated when the erythroid progenitor is matured with ubiquitin ligase activity hindered or prevented, as compared with the test protein amount when the erythroid progenitor is matured without ubiquitinase activity hindered or prevented; b) identifying if the test protein is labelled when a labelled ubiquitin construct is provided during maturation of the erythroid progenitor; or (c) identifying if the test protein is ubiquitinated through anti-ubiquitin antibody labelling or by mass spectrometry.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1A shows an example of endogenous thymidine phosphorylase (TP) expression measured by flow cytometry in isolated CD34+ stem cells, isolated reticulocytes and isolated red blood cells from blood donations whereby the dark grey depicts the IgG isotype control and light grey the TP expression. FIG. 1B shows quantification of the flow cytometry graphs (N=3). FIG. 1C shows TP activity as measured in isolated CD34+ stem cells, isolated reticulocytes, isolated red blood cells and as a control isolated platelet from blood donations, tested by a spectrophotometer assay. FIG. 1D shows an example of endogenous thymidine phosphorylase (TP) expression measured by flow cytometry in in vitro cultured day 8 (proerythroblast), day 12 (basophilic erythroblast) and reticulocytes whereby the dark grey depicts the IgG isotype control and light grey is the TP expression. FIG. 1E shows quantification of the flow cytometry graphs (N=3). FIG. 1F shows TP activity as measured in in vitro cultured day 8 (proerythroblast), day 12 (basophilic erythroblast) and reticulocytes. FIG. 1G shows an example of endogenous thymidine phosphorylase (TP) expression measured by flow cytometry in expanding BEL-A, Day 6 differentiated (polychromatic) and BEL-A derived reticulocytes whereby the dark grey depicts the IgG isotype control and light grey the TP expression. FIG. 1H shows quantification of the flow cytometry graphs (N=3).

FIG. 2 shows the increase in TP expression by exogenous overexpression of TP in cultured CD34+ derived erythroid progenitors expressing TP cells (cTP; FIG. 2A) and expanding BEL-A expressing TP cells (bTP; FIG. 2B), the TP activity in units per cTP cells (FIG. 2C) and the deformability of cTP derived reticulocytes (FIG. 2D)

FIG. 3 shows the effect of degradation inhibitors on TP expression in cTP cells (FIG. 3A) and a 3D model showing the result of modelling of human TP and location of ubiquitination sites which would need to be mutated (FIG. 3B);

FIG. 4 shows expression levels of mutated TP in cTP cells (FIG. 4A; cTP-mut) and bTP cells (FIG. 4B; bTP-mut) and activity levels of mutated TP in cTP cells (FIG. 4C; cTP-mut) and bTP cells (FIG. 4D; bTP-mut);

FIG. 5 shows expression levels of TP in the presence of thymidine supplementation in cTP cells (FIG. 5A) and bTP cells (FIG. 5B);

FIG. 6 shows a schematic of cell differentiation from a hematopoietic stem cell to a red blood cell;

FIG. 7 shows a schematic of cell differentiation lineages, starting from a hematopoietic stem cell and from a BEL-A pro-erythroblast respectively, exemplifying stages in vitro where a viral vector can be added.

FIG. 8 shows a bar graph showing overexpression of glutamine synthase during differentiation using lentivirus as assessed by flow cytometry across two different cultures as well as a bar graph showing glutamine synthase expression levels in the presence and absence of MG132.

FIG. 9 shows a bar graph showing overexpression of adenosine deaminase (tagged with c-Myc) during differentiation using lentivirus as assessed by flow cytometry across two different cultures as well as a Western blot showing expression in in vitro derived reticulocytes.

FIG. 10 shows a bar graph showing overexpression of L-asparaginase (tagged with c-Myc) using lentivirus during differentiation as assessed by flow cytometry across two different cultures as well as a Western blot showing expression in in vitro derived reticulocytes.

FIG. 11 shows a bar graph showing expression of uricase (tagged with c-Myc) using lentivirus during differentiation as assessed by flow cytometry across two different cultures as well as a Western blot showing expression in in vitro derived reticulocytes.

### DETAILED DESCRIPTION

### Example 1—Endogenous TP Expression in Erythroid Progenitors, Reticulocytes and Erythrocytes

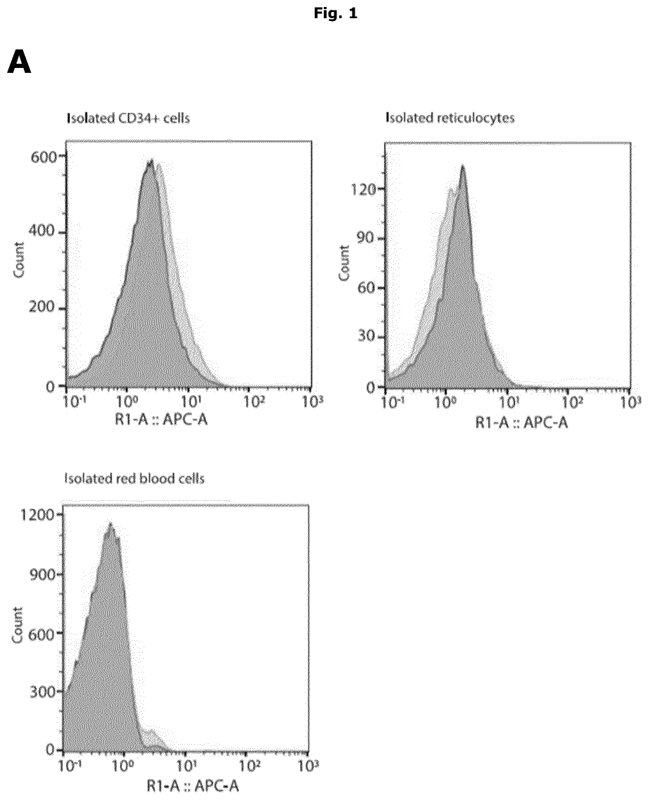

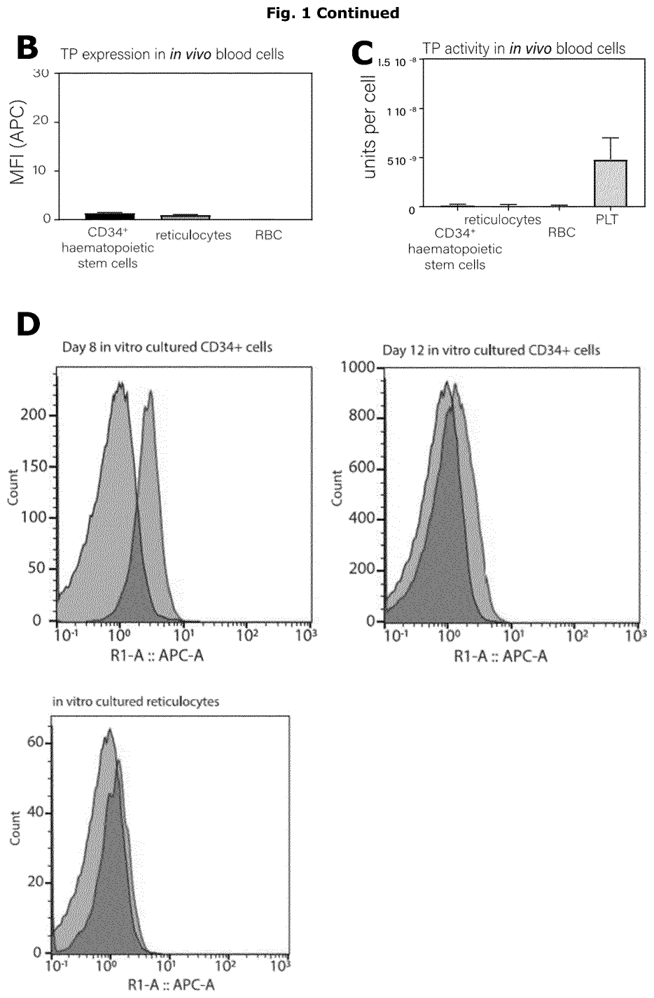

We first confirmed the base line endogenous expression and thymidine phosphorylase (TP) activity levels in isolated haematopoietic CD34⁺ stem cells (i.e. isolated from blood), standard donor derived reticulocytes and erythrocytes. Expression was assessed by flow cytometry on fixed and permeabilised cells and TP activity was determined using a spectrophotometer-based assay whereby the difference in absorbance levels of thymine, in the presence of TP, after 30 minutes at 37° C. was measured. As expected, both assays confirmed a low endogenous expression and activity of TP in CD34⁺ haematopoietic stem cells and reticulocytes and no TP expression or activity in red blood cells (FIGS. 1A, 1B and 1C). Freshly isolated platelets from peripheral blood were included as a positive control for the activity assay (1C) as these blood cells contain TP. Next, we examined the endogenous TP expression and activity in erythroid cells differentiated in vitro from CD34⁺ haematopoietic stem cells. Previously, we have reported on the different stages of erythroid maturation in our in vitro culturing system (Griffiths, R. E., et al., Blood, 2012, 119(26), p. 6296-306). We refer herein to the days in culture and their approximate stage of differentiation in brackets based on this knowledge. FIGS. 1D, 1E and 1F shows that the expression of endogenous TP and activity on day 8 (pro-erythroblast) day and day 12 (polychromatic erythroblast) is low. This also demonstrates that the expression and activity of unmodified filtered in vitro cultured reticulocytes is comparable to donor isolated endogenous reticulocytes. We next tested endogenous TP expression in the erythroid cell line, BEL-A, an erythroid cell line with the capacity to differentiate into reticulocytes, that are comparable to in vitro cultured reticulocytes (Trakarnsanga, K., et al., Nat Commun, 2017. 8: p. 14750). The advantage of using BEL-A cells is these provide a sustainable source of cells that can be genetically modified and stored frozen with the modification maintained indefinitely (Hawksworth, J., et al., EMBO Mol Med, 2018. 10(6); Trakarnsanga, K., et al., Nat Commun, 2017. 8: p. 14750), whereas CD34+ derived cultures are finite and need to be reinitiated each time. Expanding BEL-A cells (which are comparable to proerythroblast erythroblasts) have a low endogenous TP expression FIGS. 1G and 1H, and the BEL-A derived reticulocytes exhibit no measurable TP expression (FIG. 1H), comparable to CD34+ derived cultured reticulocytes (FIG. 1E) and in vivo isolated reticulocytes (FIG. 1B).

### Example 2—Exogenous Overexpression of Thymidine Phosphorylase in In Vitro CD34+ Cells and BEL-A Derived Cells Using Lentivirus

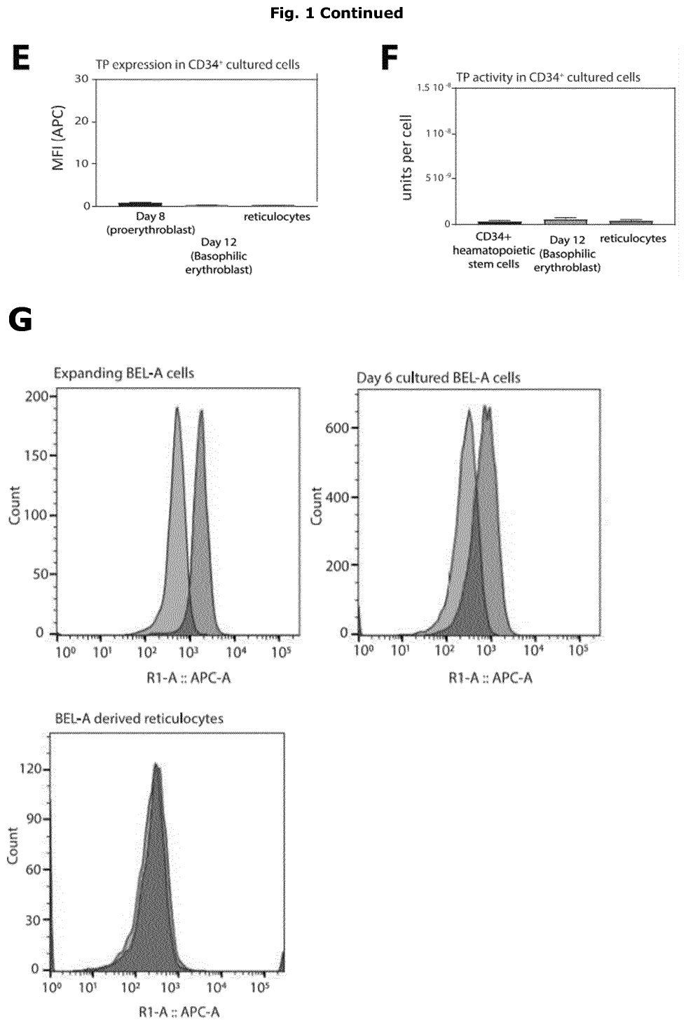

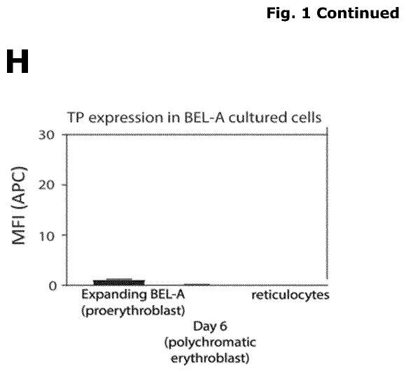

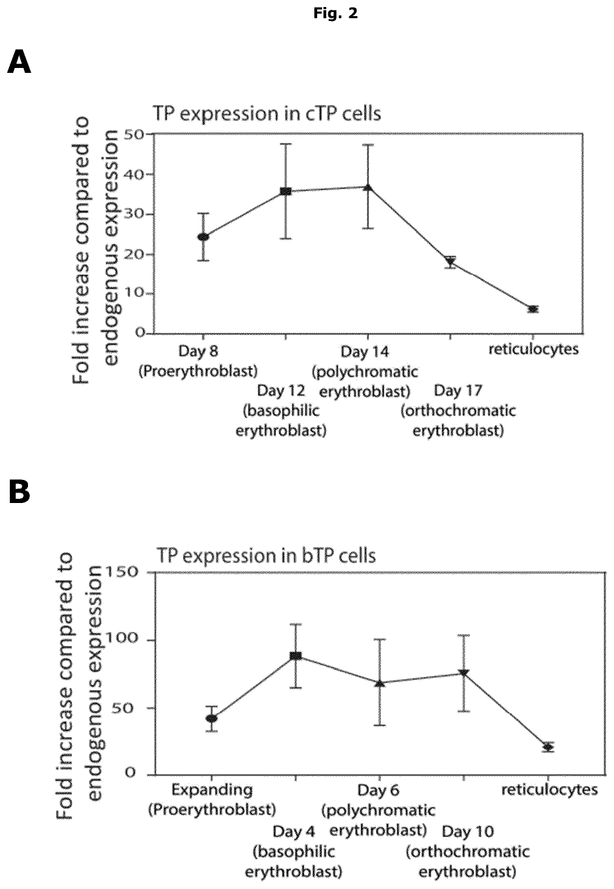

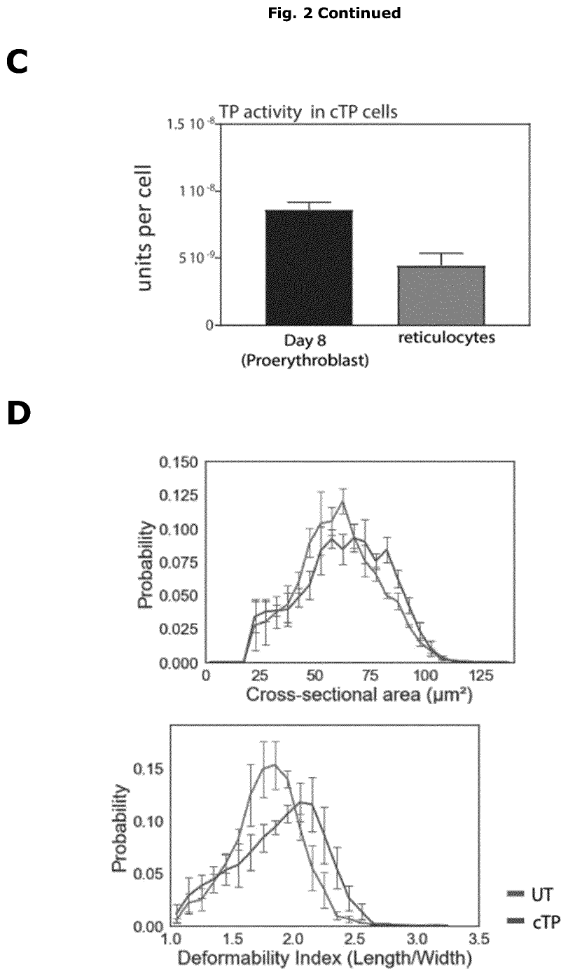

Cultured erythroid progenitors expressing TP cells (cTP) and expanding BEL-A expressing TP cells (bTP) were created by stably transducing the cells with lentivirus expressing human TP cDNA. Subclones were created from the polyclonal bTP population by blind single cell sorting using FACS. Day 6 cTP (pro-erythroblast) and expanding bTP cells (pro-erythroblast) exhibited a 25- and 45-fold increase respectively, in TP enzyme expression by flow cytometry compared to endogenous expression (FIGS. 2A and 2B). The activity assay confirmed the presence of active enzyme with an approximate concentration of 8.6×10⁻⁹ unit per polychromatic cTP cell, which is comparable to the natural endogenous expression of approximately 10 freshly isolated platelets.

The cTP cells were differentiated and TP expression was measured at day 10 (basophilic erythrocytes) day 14 (polychromatic erythrocytes) day 16 (orthochromatic erythrocytes) and in filtered reticulocytes (see FIG. 2A). bTP expression was measured during differentiation at day 4 (basophilic erythrocytes) day 6 (polychromatic erythrocytes) day 10 (orthochromatic erythrocytes) and in reticulocytes (see FIG. 2B). Though expression in cTP and bTP reticulocytes was observed to be 6 and 12-fold increased compared to endogenous levels, a striking decline of expression is observed during terminal differentiation. The TP activity measured in filtered cTP reticulocytes was 4.4×10⁻⁹U/Cell (see FIG. 2C). The deformability of the cTP derived reticulocytes was measured using an Automated Rheoscope Cell analyser (ARCA). This showed that cTP reticulocytes are comparable to the unmodified cTP control reticulocytes in both size and deformability (FIG. 2D).

### Example 3—Thymidine Phosphorylase is Degraded by the Ubiquitin Degradation Pathway in Erythroblasts

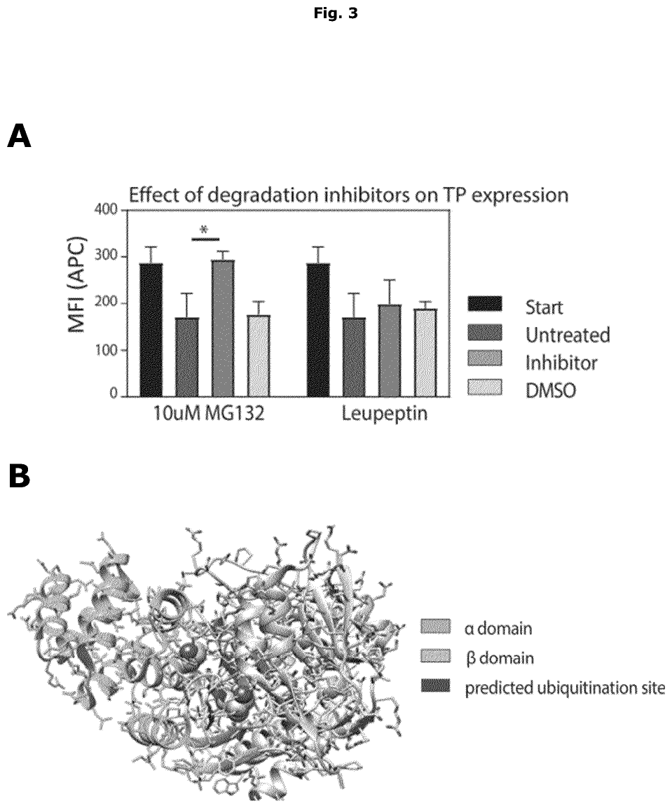

The substantial loss of TP enzyme expression during differentiation could mean that TP is being actively degraded during terminal differentiation. To test whether exogenous TP degradation during differentiation is due to ubiquitination or lysosomal degradation, we subjected the day 14 (orthochromatic erythrocytes) cTP cells to an ubiquitination inhibitor MG132, or a lysosomal degradation inhibitor, leupeptin (Tsubuki, S., et al., J Biochem, 1996, 119(3), p. 572-6; Hershko, A. and A. Ciechanover, Annu Rev Biochem, 1982, 51, p. 335-64). TP expression was first measured on day 14 and then 24 hours after incubation with inhibitors or the vehicle control. While leupeptin did not impair degradation, inhibition of degradation was observed upon addition of MG132 (FIG. 3A). This indicates that ubiquitination during differentiation is a significant cause of human TP protein degradation.

### Example 4—Modelling of Human TP and Mutagenesis of the Ubiquitination Site

Examination of the human crystal structure of the TP dimer (2J0F.pdb), showed that the protein is composed of two homodimers, each consisting of an 6 α-helixes α-domain and an α/β-domain which consists of antiparallel β-sheet surrounded by α-helixes (Norman, R. A., et al., Structure, 2004, 12(1), p. 75-84). These domains can rotate 8° relative to each other upon substrate binding. Without substrate present, TP is in an open conformation, while binding to thymidine and phosphate causes the enzyme to close. Kinetic studies conducted on E. coli and rabbit TP protein have shown that there is a sequential mechanism of binding, whereby the substrate thymidine is the first to bind, and 2-dR-1-P the last to be released (Krenitsky, T. A., J Biol Chem, 1968, 243(11), p. 2871-5).

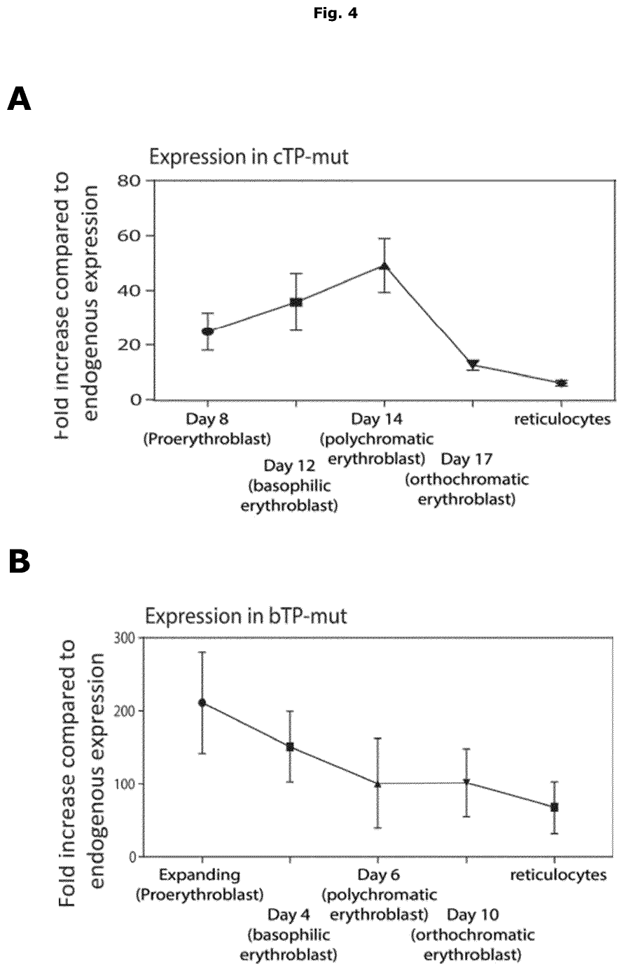

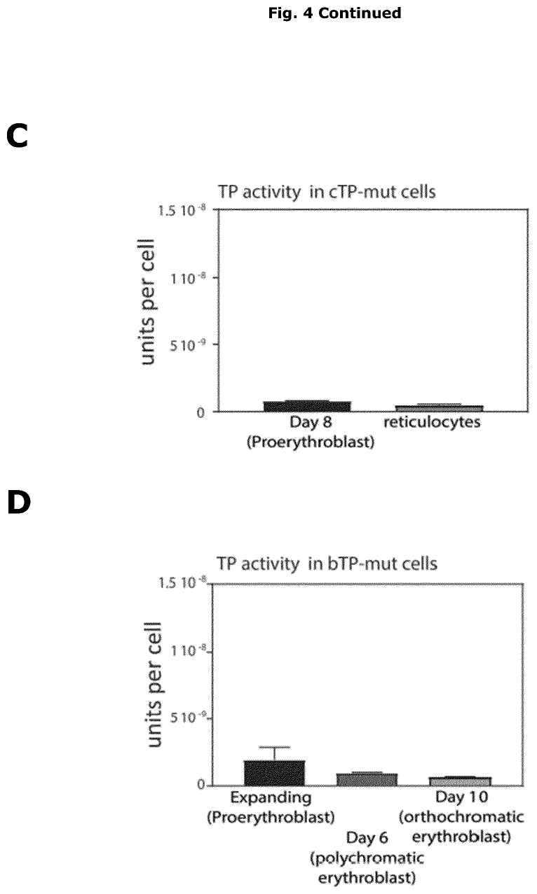

Human and mouse TP proteins are 81.2% identical and sequence comparison confirmed that 2 known ubiquitination sites located at residues 115 and 221 in mouse TP enzyme are conserved in human TP structure. Examination of the structures suggested that both conserved lysines are an integral part of the thymidine binding site and therefore the alteration of these important residues could affect TP activity or stability because the active site is altered (FIG. 3B). The two lysine residues in human TP were exchanged for arginine residue to preserve the active site structure but remove the ubiquitation sites, making TP-mut. This enzyme was expressed in both day 3 in vitro cultured erythrocytes (cTP-mut) and expanding BEL-A cells (bTP-mut) and the cells subsequently differentiated. The TP-mut expression levels achieved were comparable to day 6 cTP and expanding bTP cells (see FIGS. 4A and 4B) but no TP activity was detected in the cTP-mut reticulocyte (FIG. 4C). The mutations compromise the enzyme activity and stability. This provides proof of concept that mutation of ubiquitination sites can be used to prevent protein degradation through the enucleation process, but that care must be taken to ensure any mutation does not destroy the desired protein activity. Therefore, either the enzyme active site would need to be reengineered to retain activity but remove the ubiquitin sites or an alternate means of disrupting TP ubiquitination are required.

### Example 5—Thymidine Phosphorylase Degradation is Reduced by Thymidine Supplementation

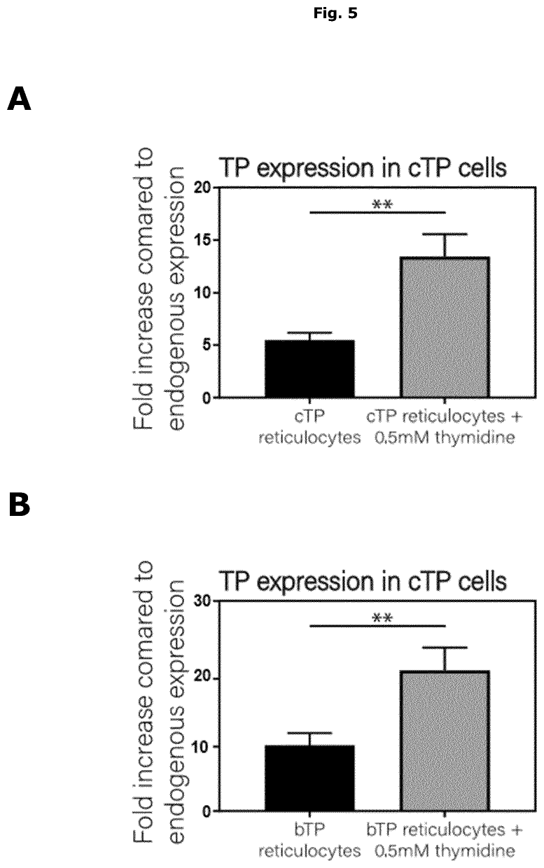

After further examination of the molecular human TP structure we observed that the 2 ubiquitination sites that correspond with murine TP, would only be available for ubiquitination in the absence of substrate. We therefore hypothesised that supplementation of the culture media with TP enzyme substrate thymidine may result in a closed TP structure, reducing its degradation by obscuring access to the lysine's ubiquitination sites in the active site. However, previous reports have shown that addition of thymidine can arrest cell cycle and also inhibited cell growth in K562 cells at a concentration of 1 mM (Anisimov, A. G., et al., Izv Akad Nauk Ser Biol, 2003(3), p. 275-84; Thomas, D. B. and C. A. Lingwood, Cell, 1975, 5(1), p. 37-42). To determine if increased thymidine concentrations in our culture media could prevent TP degradation, media was daily supplemented with 0.5 mM thymidine starting at different points during the culture process. We confirmed an increase in cell death and inhibition of differentiation when thymidine was added early during in vitro culture e.g. Day 0 of differentiation, data not shown. To circumvent this, 0.5 mM thymidine supplementation was added daily during differentiation, from day 14 (polychromatic erythroblast stage) in cTP cells and day 6 (polychromatic erythroblast stage) in bTP cells of differentiation, which is approximately the point overexpressed human TP is normally degraded. This manoeuvre doubled TP enzyme abundance in both cTP reticulocytes and bTP reticulocytes compared to cells produced using the standard differentiation media (FIGS. 5A and 5B) which was not supplemented with thymidine.

### Example 6—Investigating Retention of Further Enzymes

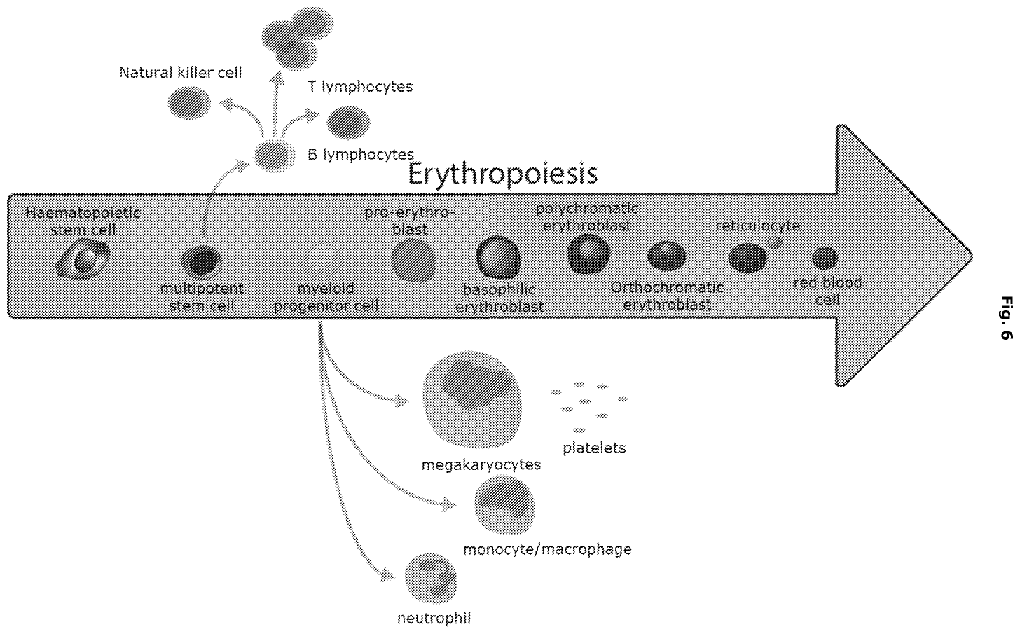

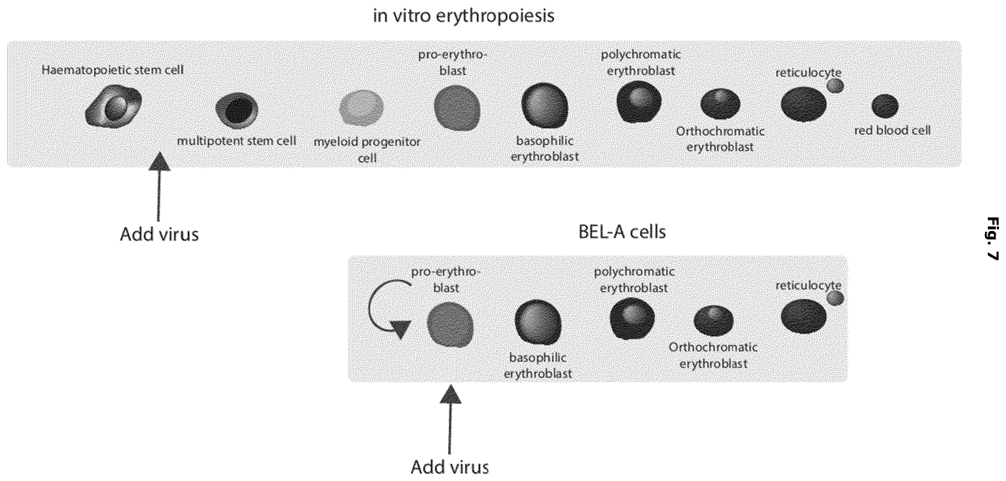

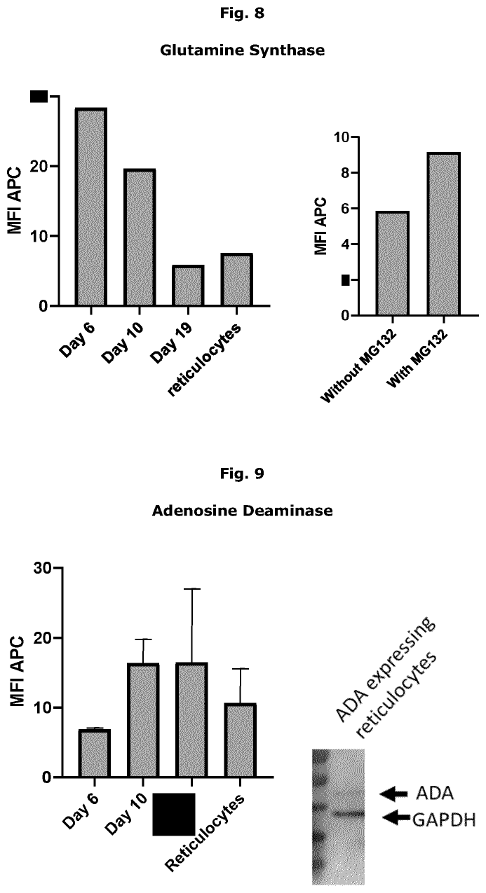

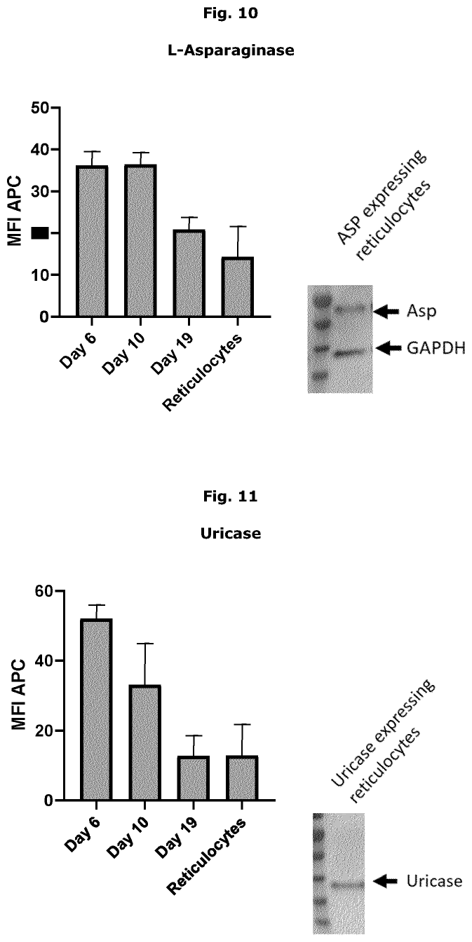

To investigate the degree to which exogenous enzyme expression is retained during erythropoiesis, four different enzymes were expressed in CD34+ haematopoietic stem cells (using lentivirus) and subsequently differentiated to reticulocytes: glutamine synthase (FIG. 8), adenosine deaminase (FIG. 9), L-asparaginase (FIG. 10), and uricase (FIG. 11). The exogenous proteins Uricase (a bacterial enzyme), human L-asparaginase (ASP) and human adenine deaminase (ADA) were tagged with c-Myc to facilitate measurement of expression during differentiation by flow cytometry and western blot. GAPDH is an endogenous enzyme which was also blotted for in some cases. For human glutamine synthetase (GS) an antibody specific for this protein was used to detect expression by flow cytometry. The bar graphs in FIGS. 8-11 show expression of indicated enzymes during differentiation as assessed by flow cytometry across two different cultures. Western blots illustrate expression of the enzymes in in vitro derived reticulocytes. To explore whether ubiquitination enhances GS enzyme retention at the end of the culture, MG132 (a ubiquitin inhibitor) was added at day 15 to the GS (over)expressing erythroblasts. Expression was measured at day 19 by flow cytometry.

### Example 7—Identification of Further Enzymes Compatible with Invention

The inventors conducted sequence site scans for ubiquitin consensus sequences on key enzymes according to the method below. For alanine aminotransferase, site scan for consensus sequences suggest ubiquitin sites on both human isoforms. For glutamate dehydrogenase, site scan suggests ubiquitin sites on one of two enzymes. For arginine deiminase, there are 6 human isoforms and site scan identified that 3 isoforms as having ubiquitin consensus sites. For arginase, one of two isoforms was identified as having a ubiquitin consensus site.

### Material and Methods

Antibodies

Monoclonal thymidine phosphorylase antibody (clone P-GF.44C) was used at 1/10 dilution (Thermo scientific). Secondary antibodies used were APC-conjugated monoclonal anti-mouse IgG1 or polyclonal anti-IgG (Biolegend) or Alexa647-anti-human (Jackson Laboratories) and used at 1:50 (v/v).

BEL-A Cell Culture

BEL-A cells were cultured as previously described (Trakarnsanga, K., et al., Nat Commun, 2017, 8, p. 14750). In brief, cells were maintained in expansion medium StemSpan SFEM (Stem Cell Technologies) supplemented with 50 ng/mL SCF milteny, 3 U/mL EPO (Roche, Welwyn Garden City, UK), 1 μM dexamethasone (Sigma-Aldrich) and 1 μg/mL doxycycline (Sigma-Aldrich)] at 1-3×105 cells/mL. Complete medium changes were performed every 48 hours. Differentiation was induced as previously described: cells were seeded at 1.5×10⁵/mL in differentiation medium (Iscove's modified Dulbecco's medium (IMDM), Source BioScience, Nottingham UK) containing 3% (v/v) AB Serum (Sigma-Aldrich, Poole UK), 2 mg/ml HSA (Irvine Scientific, Newtown Mount Kennedy, Ireland), 10 μg/ml Insulin (Sigma-Aldrich), 3U/ml heparin (Sigma-Aldrich), 500 μg/ml transferrin (Sanquin Blood Supply, Netherlands) and 3U/ml Epo (Roche, Welwyn Garden City, UK) supplemented with 1 ng/mL IL-3 (R&D Systems, Abingdon UK), 10 ng/mL SCF and 1 μg/mL doxycycline. After 2 days, cells were reseeded at 3×10⁵/ml in fresh medium. On differentiation day 4, cells were reseeded at 5×10⁵/ml in fresh medium without doxycycline. On differentiation day 6, a complete media change was performed, and cells were reseeded at 1×10⁶/mL. On day 8, cells were transferred to differentiation medium (containing no SCF, IL-3 or doxycycline) and maintained at 1×10⁶/mL with complete medium changes every 2 days until day 12.

CD34 Cell Culture

As previously described (Griffiths, R. E., et al., Blood, 2012. 119(26), p. 6296-306), CD34+ haemopoietic stem cells (HSC) here isolated from human blood donor mononuclear cells or from thawed cryopreserved cord blood units by magnetic bead separation according to manufacturer's instructions (Miltenyi Biotech Ltd, Bisley UK). CD34+ cells were grown at a density of 2×10⁵ cells/ml using a base medium consisting of IMDM (Source BioScience) containing 3% (v/v) AB Serum, 2 mg/ml HSA, 10 μg/ml Insulin, 3U/ml heparin, 500 μg/ml transferrin and 3U/ml Epo. In the first stage (days 0-10) this was supplemented with 10 ng/ml stem cell factor and 1 ng/ml IL-3 and in the second stage (days 11-13) with 10 ng/ml SCF. In the final stage to day 19, only the base medium was used. 10 μM leupeptin (Sigma-Aldrich) or 5 μM MG132 (Sigma-Aldrich) was added to 1×10⁶ differentiating erythroblasts for 24 hours at 37° C., 5% CO₂. Cells where fixed with paraformaldehyde and analysed by flow cytometry.

Lentiviral Transduction

Human TP cDNA sequence was ordered and cloned in the XLG3 vector by Genscript (Genscript, Leiden NL). The original TP sequence was mutated at sites 115 and 221 from a lysine to arginine, thereby creating the TP-mut Lentivirus was prepared according to previously published protocols (Satchwell et al Haematologica, 2015 100; 133-142 Doi:10.3324/haematol.2014.114538). For transduction of BEL-A and CD34+ haematopoietic cells, virus was added to 2×10⁵ cells in 2 ml medium in the presence of 8 μg/ml polybrene for 24 h. Cells were washed three times and resuspended in fresh medium.

Flow Cytometry and FACS

For flow cytometry on undifferentiated BEL-As, 1×10⁵ cells were fixed 1% paraformaldehyde, 0.0075% glutaraldehyde and permeabilised with 0.1% Triton X-100, resuspended in PBSAG (PBS+1 mg/ml BSA, 2 mg/ml glucose)+1% BSA were labelled with primary antibody for 30 minutes at 4° C. Cells were washed in PBSAG, incubated for 30 min at 4° C. with appropriate APC-conjugated secondary antibody, and washed and data acquired on a MacsQuant VYB Analyser using a plate reader. Reticulocytes were identified by gating upon Hoechst-negative population. For FACS sorting of cells, a BDInflux Cell Sorter was used to isolate single clones, by sorting the propidium iodide-negative population into 96-well plates.

TP Activity Assay

The cells were resuspended 1×10⁶ cells in lysis buffer (50 mM Tris-HCl, pH 7.2, 1% (w/v) triton X-100, 2 mM phenylmethylsulfonyl fluoride (PMSF), 0.02% (v/v) 2-mercaptoethanol). The lysate was centrifuged at 16000 g for 30 min at 4° C. and then add 176 mM thymidine and 5× TP reaction buffer (0.5 M Tris-arsenate, pH 6.5) to the supernatant. The controls used were lysis buffer alone or lysis buffer containing known concentrations of purified TP protein (Sigma-Aldrich, Poole UK). The reaction was incubated at 37° C. for 30 min and then terminated by addition of 0.3M NaOH. The absorbance measured using a spectrophotometer at 299 nm and compared to the TP enzyme standard concentration curve. (Martí, R., L. C. López, and M. Hirano, Methods in Molecular Biology (Clifton, N.J.), 2012. 837: p. 121-133)

Reticulocyte Deformability Measurements Using ARCA

1×10⁶ reticulocytes were resuspended in 200 μl polyvinylpyrrolidone solution (PVP viscosity 28.1; Mechatronics Instruments, The Netherlands). Samples were assayed in an ARCA (Dobbe, J. G. G., et al., Measurement of the distribution of red blood cell deformability using an automated rheoscope. 2002. 50(6), p. 313-325) which consists of a plate-plate optical shearing stage (model CSS450) mounted on a Linkam imaging station assembly and temperature controlled using Linksys32 software (Linkam Scientific Instruments, Surrey, UK). The microscope was equipped with an LMPlanFL 50× with a 10.6 mm working distance objective (Olympus, Essex, UK) illuminated by a X-1500 stroboscope (PerkinElmer, The Netherlands) through a band-pass interference filter (CWL 420 nm, FWHM 10 nm; Edmund Optics, Poppleton, UK). Images were acquired using a uEye camera (UI-2140SE-M-GL; IDS GmbH, Obersulm, Germany). At least 1,000 cell images per sample were acquired and analysed using bespoke ARCA software.

TP Modelling

Ubiquitination sites were predicted from mUbiSiDa—Database of mammalian protein ubiquitination sites (http://202.195.183.4:8000/BroGo3_data.php?name=0016154). Which provided details of the mouse ubiquitination sites analysed in Wagner et al (Mol Cell Proteomics, 2012, 11(12), p. 1578-85). Clustal Omega (DOI: 10.1093/nar/gkz268) was used to align human and mouse sequences. Protein structures were visualized, and image produced with UCSF chimera software (DOI: 10.1002/jcc.20084).

Ubiquitin site consensus sequence identification. The full protein sequence was subjected to a search for ubiquitin consensus sequences using two sequence search programs. The first is UbPred (Radivojac, P., Vacic, V., Haynes, C., Cocklin, R. R., Mohan, A., Heyen, J. W., Goebl, M. G., and Iakoucheva, L. M. Identification, Analysis and Prediction of Protein Ubiquitination Sites. Proteins: Structure, Function, and Bioinformatics. 78(2):365-380. (2010)) which can be accessed online at http://www.ubpred.org/ and the second is PhosphoSite (“Hornbeck P V, Zhang B, Murray B, Kornhauser J M, Latham V, Skrzypek E PhosphoSitePlus, 2014: mutations, PTMs and recalibrations. Nucleic Acids Res. 2015 43:D512-20.”) which can be accessed online at https://www.phosphosite.org/ (the latter provides all potential post translational sites—including acetylation and ubiquitination).

1. A method of making a reticulocyte comprising elevated levels of a target protein or polypeptide, the method comprising:
a) provision of an erythroid progenitor which is able to express the target protein or polypeptide;
b) expression of the target protein or polypeptide; and
c) maturation of the erythroid progenitor into the reticulocyte;
wherein during maturation of the erythroid progenitor into the reticulocyte, the target protein or polypeptide is configured and/or inhibited such that ubiquitination of the target protein or polypeptide is hindered or prevented.

2. A method according to claim 1, wherein ubiquitination of the target protein or polypeptide is hindered or prevented by:
i. where the target protein or polypeptide comprises a ubiquitination site, provision of an inhibitor of the target protein or polypeptide ubiquitination site during maturation of the erythroid progenitor into the reticulocyte;
ii. where the target protein or polypeptide is a variant of a source protein or polypeptide with a ubiquitination site, provision of an erythroid progenitor which is able to express a target protein or polypeptide comprising a mutation that renders ubiquitination hindered or prevented with respect to the source protein or polypeptide; and/or
iii. provision of an erythroid progenitor which is able to express an exogenous protein or polypeptide having no ubiquitination site.

3. A method according to claim 1, comprising the further step of:
d) red cell development, in particular maturation of the reticulocyte into an erythrocyte;
wherein during maturation to the erythrocyte, the target protein or polypeptide is configured and/or inhibited such that ubiquitination is hindered or prevented, preferably hindered or prevented by any of (i)-(iii) of claim 2.

4. A method according to any preceding claim, wherein the target protein comprises an endogenous protein and/or the target polypeptide comprises an endogenous polypeptide.

5. A method according to any of claims 1-3, wherein the target protein comprises an overexpressed endogenous protein or an exogenous protein and/or the target polypeptide comprises an overexpressed endogenous polypeptide or an exogenous polypeptide.

6. A method according to any preceding claim, wherein the inhibitor is a natural substrate or a natural product of the target protein or polypeptide or is a reversible inhibitor of a natural substrate or natural product of the target protein or polypeptide.

7. A method according to any preceding claim, wherein the erythroid progenitor is a stem cell, haematopoietic stem cell, induced pluripotent cell, erythroid immortalized cell line or erythroblast cell, preferably a CD34+ cell, CD34− cell, or a BEL-A cell.

8. A method according to any preceding claim, wherein the target protein or polypeptide is an enzyme.

9. A method according to any preceding claim, wherein the target protein is thymidine phosphorylase, glutamine synthase, hexokinase/glucokinase, phenylalanine hydroxylase, alcohol dehydrogenase, catalase, glucose-6-phosphate dehydrogenase, adenosine deaminase, L-asparaginase, uricase, bacterial L-phenylalanine ammonia lyase, alanine aminotransferase, glutamate dehydrogenase, arginine deiminase, or arginase.

10. A method according to any preceding claim, wherein the target protein is thymidine phosphorylase, and the thymidine phosphorylase ubiquitination site is inhibited by thymidine, deoxyuridine, thymine, uridine, 2-deoxy ribose 1-phosphate, or derivatives or analogs of these, preferably thymidine.

11. A method according to any of claims 2 to 10, wherein the exogenous protein or polypeptide having no ubiquitination site is a non-eukaryotic protein or polypeptide, preferably a bacterial protein or polypeptide.

12. A method according to any preceding claim, wherein the reticulocyte or erythrocyte is an isolated reticulocyte or isolated erythrocyte.

13. An erythroid cell comprising a target protein or polypeptide, wherein:
i) where the target protein or polypeptide comprises a ubiquitination site, the erythroid cell further comprises an inhibitor of the target protein or polypeptide ubiquitination site; and/or
ii) where the target protein or polypeptide is a variant of a source protein or polypeptide with a ubiquitination site, the target protein or polypeptide comprises a mutation that renders ubiquitination hindered or prevented with respect to the source protein or polypeptide; and/or
iii) the target protein or polypeptide comprises an exogenous protein or polypeptide having no ubiquitination site.

14. An erythroid cell according to claim 13, wherein the erythroid cell is an erythroid progenitor cell.

15. An erythroid cell according to claim 13, wherein the erythroid cell is an enucleated erythroid cell.

16. An erythroid cell according to any of claims 13-15, wherein the target protein comprises an endogenous protein and/or the target polypeptide comprises an endogenous polypeptide.

17. An erythroid cell according to any of claims 13-15, wherein the target protein comprises an overexpressed endogenous protein or an exogenous protein and/or the target polypeptide comprises an overexpressed endogenous polypeptide or an exogenous polypeptide.

18. An erythroid cell according to any of claims 13-17, wherein the inhibitor is a natural substrate or a natural product of the target protein or polypeptide or is a reversible inhibitor of a natural substrate or natural product of the target protein or polypeptide.

19. An erythroid cell according to any of claims 13-18, wherein the target protein is an enzyme.

20. An erythroid cell according to any of claims 13-19, wherein the target protein is thymidine phosphorylase, glutamine synthase, hexokinase/glucokinase, phenylalanine hydroxylase, alcohol dehydrogenase, catalase, glucose-6-phosphate dehydrogenase, adenosine deaminase, L-asparaginase, uricase, bacterial L-phenylalanine ammonia lyase, alanine aminotransferase, glutamate dehydrogenase, arginine deiminase, or arginase.

21. An erythroid cell according to any of claims 13-20, wherein the target protein is thymidine phosphorylase and the thymidine phosphorylase ubiquitination site is inhibited by thymidine, deoxyuridine, thymine, uridine and/or 2-deoxy ribose 1-phosphate, or derivatives or analogues of these, preferably thymidine.

22. An erythroid cell according to any of claims 13-21, wherein the erythroid cell is an isolated erythroid cell.

23. An erythroid cell according to any of claims 13-22, wherein the target protein or polypeptide that has no ubiquitination site is a non-eukaryotic protein, preferably a bacterial protein.

24. An erythroid cell obtainable by the method of any of claims 1-12.

25. A pharmaceutical composition comprising an erythroid cell according to any one of claims 13-24 and a pharmaceutically acceptable carrier, excipient and/or adjuvant.

26. A pharmaceutical composition according to claim 25, comprising an erythroid cell stored in an erythroid cell storage buffer comprising the inhibitor of the target protein or polypeptide ubiquitination site.

27. A pharmaceutical composition comprising an erythroid cell comprising a target protein or polypeptide comprising a ubiquitination site and an inhibitor of the target protein or polypeptide ubiquitination site and a pharmaceutically acceptable carrier, excipient and/or adjuvant.

28. A pharmaceutical composition according to claim 27, wherein the erythroid cell is stored in an erythroid cell storage buffer comprising the inhibitor of the target protein or polypeptide ubiquitination site.

29. A pharmaceutical composition comprising an erythroid cell comprising a target protein or polypeptide, wherein the target protein or polypeptide is a variant of a source protein comprising a ubiquitination site, and wherein the target protein or polypeptide comprises a mutation that renders ubiquitination hindered or prevented with respect to the source protein or polypeptide and a pharmaceutically acceptable carrier, excipient and/or adjuvant.

30. A pharmaceutical composition according to claim 28 or 29, wherein the erythroid cell is an enucleated erythroid cell, preferably a reticulocyte or erythrocyte.

31. An erythroid cell according to any of claims 13-24, or a pharmaceutical composition according to any of claims 25-30, for use in therapy.

32. An erythroid cell or a pharmaceutical composition for use according to claim 31, for use in enzyme replacement therapy, organ reconditioning or detoxification, preferably for the treatment of Mitochondrial Neurogastrointestinal Encephalomyopathy, Hyperammonemia, Hyperglycaemia, phenylalanine hydroxylase enzyme deficiency, alcohol toxicity/detoxification, catalase deficiency and/or preventing cell damage by reactive oxygen species, G6PD deficiency, adenosine deaminase deficiency, acute lymphoblastic leukemia cancer, hyperuricemia, phenylketonuria/phenylalanine hydroxylase enzyme deficiency, or hyperargininaemia.

33. Use of an erythroid cell according to any of claims 13-24, or a pharmaceutical composition according to any of claims 25-30, for the manufacture of a medicament for use in therapy.

34. A method of treatment of a subject in need thereof, the method comprising administering a pharmaceutically effective amount of an erythroid cell according to any of claims 13-24, or a pharmaceutically effective amount of a pharmaceutical composition according to any of claims 25-30.

35. A method of screening for a protein or polypeptide that is degraded by ubiquitination during maturation of an erythroid progenitor, the method comprising expressing a test protein in an erythroid progenitor and:
(a) identifying if the test protein amount is elevated when the erythroid progenitor is matured with ubiquitinase activity hindered or prevented, as compared with the test protein amount when the erythroid progenitor is matured without ubiquitinase activity hindered or prevented;
(b) identifying if a test protein is labelled when a labelled ubiquitin construct is provided during maturation of the erythroid progenitor; or
(c) identifying if the test protein is ubiquitinated through anti-ubiquitin antibody labelling or by mass spectrometry.
